(19) United States
(12) Patent Application Publication
(10) Pub. No.: US 2001/0053522 A1
(43) Pub. Date: Dec. 20, 2001

(54) DNA chip and reactive electrode

(75) Inventors: Yoshihiko Makino, Saitama (JP); Yoshihiko Abe, Saitama (JP); Masashi Ogawa, Tokyo (JP)

Correspondence Address: Jules Goldberg, Jules E. Goldberg, Esq., REED SMITH LLP, 375 Park Avenue, New York, NY, 10152, US

(73) Assignee: Fuji Photo Film Co., Ltd.

(21) Appl. No.: 09/845,403

(22) Filed: Apr. 30, 2001

(30) Foreign Application Priority Data
Apr. 28, 2000 (JP) 2000-130090

(51) Int. Cl. C12Q 1/68

(52) U.S. Cl. 435/6; 438/1

(57) ABSTRACT

A nucleic acid detective means composed of an electrode and plural peptide nucleic acids which are fixed onto the electrode via covalent bonding is favorably employed for electrochemically detecting complementary DNA fragments The covalent bonding between the electrode and the peptide nucleic acids are favorably produced by the reaction between a reactive hydrogen-containing group attached to the peptide nucleic acid and a vinylsulfonyl group or a reactive precursor thereof attached to the electrode.

### FIELD OF THE INVENTION

This invention relates to a new detective means favorably employable for electrochemically detecting a complementary nucleic acid fragment, that is similar to the DNA chip.

### BACKGROUND OF THE INVENTION

Detection of a nucleic acid fragment is generally performed using a probe oligonucleotide which is complementary to the nucleic acid fragment to be detected, by way of hybridization. The probe oligonucleotide is generally fixed onto-a solid carrier (e.g., solid substrate) to produce a so-called DNA chip. In the detection procedures, a nucleic acid fragment in a sample liquid is provided with a fluorescent label or a radioisotope label, and then the sample liquid is brought into contact with the probe oligonucleotide of the DNA chip. If the labelled nucleic acid fragment in the sample liquid is complementary to the probe oligonucleotide, the labelled nucleic acid fragment is combined with the probe oligonucleotide by hybridization. The labelled nucleic acid fragment fixed to the DNA chip by hybridization with the probe oligonucleotide is then detected by an appropriate detection method such as fluorometry or autoradiography. The DNA chip is widely employed in the gene technology, for instance, for detecting a complementary nucleic acid fragment and sequencing a nucleic acid.

The DNA chip can be utilized to efficiently detect a large number of complementary nucleic acid fragments in a small amount of a sample liquid almost simultaneously.

Detection of nucleic acid fragments using an electrochemical label is also known (preprint of the 57th Analytical Chemistry Conference pp. 137-138 (1996)).

P. E. Nielsen et al., Science, 254, 1497-1500(1991) and P. E. Nielsen et al., Biochemistry, 36, pp.5072-5077 (1997) describe PNA (Peptide Nucleic Acid or polyamide Nucleic Acid) which has no negative charge and functions in the same manner as DNA fragment does. PNA has a polyamide skeleton of N-(2-aminoethyl)glycine units and has neither glucose units nor phosphate groups.

Since PNA is electrically neutral and is not charged in the absence of an electrolytic salt, PNA is able to hybridize with a complementary nucleic acid fragment to form a hybrid which is more stable than the hybrid structure given by a probe oligonucleotide and its complementary nucleic acid fragment (Preprint of the 74th Spring Conference of Japan Chemical Society, pp. 1287, reported by Naomi Sugimoto).

Japanese Patent Provisional Publication No.11-332595 describes a PNA probe fixed onto a solid carrier at its one end and a detection method utilizing the PNA probe. The PNA probe is fixed onto the solid carrier by the known combination of avidin and biotin.

The aforementioned P. E. Nielsen et al., Science, 254, 1497-1500(1991) also describes a PNA probe labelled with an isotope element and a detection method of a complementary nucleic acid fragment.

Since the PNA probe shows no electric repulsion to a target nucleic acid fragment in a sample liquid, an improved high detection sensitivity is expected.

At present, two methods are known for preparing a DNA chip having a solid carrier and oligonucleotides or polynucleotides fixed onto the carrier. One preparation method comprises preparing oligonucleotides or polynucleotides step by step on the carrier. This method is named “on-chip method”. A typical on-chip method is described in Foder, S. P. A., Science, 251, page 767 (1991).

Another preparation method comprises fixing separately prepared oligonucleotides or polynucleotides onto a solid carrier. Various methods are known for various oligonucleotides and polynucleotides.

In the case that the complementary nucleotide derivatives (which are synthesized using mRNA as mold) or PCR products (which are DNA fragments prepared by multiplying cDNA by PCR method), an aqueous solution of the prepared DNA fragment is spotted onto a solid carrier having a poly-cationic coat in a DNA chip-preparing device to fix the DNA fragment to the carrier via electrostatic bonding, and then blocking a free surface of the polycationic coat.

In the case that the oligonucleotides are synthetically prepared and have a functional group, an aqueous solution of the synthetic oligonucleotides is spotted onto an activated or reactive solid carrier to produce covalent bonding between the oligonucleotides and the carrier surface. See Lamture, J. B., et al., Nucl. Acids Res., 22, 2121-2125, 1994, and Guo, Z., et al., Nucl. Acids Res., 22, 5456-5465, 1994. Generally, the oligonucleotides are covalently bonded to the surface activated carrier via linking groups.

Also known is a process comprising the steps of aligning small polyacrylamide gels on a glass plate and fixing synthetic oligonucleotides onto the glass plate by making a covalent bond between the polyacrylamide and the oligonucleotide (Yershov, G., et al., Proc. Natl. Acad. Sci. USA, 94, 4913(1996)). Sosnowski, R. G., et al., Proc. Natl. Acad. Sci. USA, 94, 1119-1123 (1997) discloses a process comprising the steps of an array of microelectrodes on a silica chip, forming on the microelectrode a streptoavidin-comprising agarose layer, and attaching biotin-modified DNA fragment to the agarose layer by positively charging the agarose layer. Schena, M., et al., Proc. Natl. Acadl. Sci. USA, 93, 10614-10619 (1996) teaches a process comprising the steps of preparing a suspension of an amino group-modified PCR product in SSC (i.e., standard sodium chloride-citric acid buffer solution), spotting the suspension onto a slide glass, incubating the spotted glass slide, treating the incubated slide glass with sodium borohydride, and heating thus treated slide glass.

As is explained above, most of the known methods of fixing separately prepared DNA fragments onto a solid carrier utilize the electrostatic bonding or the covalent bonding such as described above.

In any DNA chips having separately prepared oligonucleotide probes on its solid carrier, the oligonucleotide probes should be firmly fixed onto the carrier, so that the hybridization can proceed smoothly between the fixed oligonucleotide probes and target DNA fragments complementary to the fixed oligonucleotide probes.

U.S. Pat. No. 5,387,505 describes a method of separating a target DNA fragment by binding target DNA fragments labelled with a biotin molecule with a substrate having avidin molecules.

U.S. Pat. No. 5,094,962 discloses a detection tool for a ligand-receptor assay in which receptor molecules are bonded to a porous polymer particle having a reactive group.

### SUMMARY OF THE INVENTION

It is an object of the present invention to provide a detective means which is favorably employable for electrochemically detecting complementary nucleic acids such as complementary DNA.

It is another object of the invention to provide a method for electrochemically detecting complementary nucleic acids such as complementary DNA with high sensitivity and high reproducibility.

The present invention resides in a nucleic acid detective means comprising an electrode and a plurality of peptide nucleic acids which are fixed onto the electrode via covalent bonding.

In the detective means of the invention, the covalent bonding between the electrode and the peptide nucleic acids are preferably produced by the reaction between a reactive hydrogen-containing group attached to the peptide nucleic acid and a vinylsulfonyl group or a reactive precursor thereof attached to the electrode. The vinylsulfonyl group or a reactive precursor thereof is preferably represented by the following formula:

-L-SO₂—X

in which L represents a linking group, and X represents a group of —CR¹═CR²R³ or —CHR¹—CR²R³Y wherein each of R¹, R² and R³ independently is a hydrogen atom, an alkyl group having 1 to 6 carbon atoms, an aryl group having 6 to 20 carbon atoms, or an aralkyl group having 7 to 26 carbon atoms in which its alkyl group has 1 to 6 carbon atoms; Y represents a halogen atom, —SO₂R¹¹, —OCOR¹², —OSO₃M, or a quaternary pyridinium group; R¹¹ is a hydrogen atom, an alkyl group having 1 to 6 carbon atoms, an aryl group having 6 to 20 carbon atoms, or an aralkyl group having 7 to 26 carbon atoms in which its alkyl group has 1 to 6 carbon atoms; R¹² is an alkyl group having 1 to 6 carbon atoms, or a halogenated alkyl group having 1 to 6 carbon atoms; and M is a hydrogen atom, an alkali metal atom, or an ammonium group.

In the detective means of the invention, the covalent bonding between the electrode and the peptide nucleic acids may be produced by the reaction between a reactive hydrogen-containing group attached to the electrode and a vinylsulfonyl group or a reactive precursor thereof attached to the peptide nucleic acid.

The nucleic acid detective means of the invention can comprise, in addition to the electrode and peptide nucleic acids, a plurality of spacer groups which are fixed onto the electrode under the condition that the spacers are placed between the peptide nucleic acids.

The invention further resides in a method for detecting a complementary nucleic acid sample which comprises the steps of:

bringing a nucleic acid sample into contact with a detective means of the invention which comprises an electrode and a plurality of peptide nucleic acids fixed onto the electrode via covalent bonding in an aqueous medium in the presence of a threading intercalator having an electroconductive moiety to produce a hybrid of the peptide nucleic acid and nucleic acid sample in which the intercalator is intercalated;

applying an electric potential to the electrode of the detective means; and

measuring an electric current flowing between the electrode of the detective means and a separately placed counter electrode through the electroconductive moiety of the intercalator;

The detection method of the invention can comprise the steps of:

bringing a nucleic acid sample into contact with a detective means which comprises an electrode and a plurality of peptide nucleic acids fixed onto the electrode via covalent bonding in an aqueous medium in the presence of a cationic compound having an electroconductive moiety to produce a hybrid of the peptide nucleic acid and nucleic acid sample to which the cationic compound is attached;

applying an electric potential to the electrode of the detective means; and

measuring an electric current flowing between the electrode of the detective means and a separately placed counter electrode through the electroconductive moiety of the cationic compound.

In the detection method of the invention, a combination of a substrate and an oxidase which reacts with the substrate to become a reduction type can be present when the electric current is measured. The electric current flowing between the electrode and the electroconductive moiety can be multiplied by the electron transfer between the electrode and the oxidase of reduction type. In the specification, the measurement of electric current by the aid of the combination of a substrate and an oxidase is named “sensitivity increased measurement”.

The invention further resides in a reactive electrode comprising an electrode and a plurality of vinylsulfonyl groups or reactive precursors thereof attached to the electrode.

The reactive electrode of the invention can comprise, in addition to the electrode and vinylsulfonyl groups or reactive precursors thereof, a plurality of inert spacer groups which are fixed onto the electrode under the condition that the spacers are placed between the vinylsulfonyl groups or reactive precursors.

### BRIEF DESCRIPTION OF DRAWINGS

FIG. 1 schematically illustrates a representative PNA-fixed electrode and its production processes according to the invention.

FIG. 2 schematically illustrates a representative PNA-fixed electrode of another type and its production processes according to the invention.

### DETAILED DESCRIPTION OF THE INVENTION

[Electrode]

The electrode, namely, electroconductive substrate can be made of electrode material, optical fiber, photo-diode, thermistor, piezo electrical element, or surface elasticity element. The electrode material is generally employed. The electrode can be carbon electrode of graphite or glassy carbon, noble metal electrode of platinum, gold, palladium, or rhodium, metal oxide electrode of titanium dioxide, tin oxide, manganese oxide, or lead oxide, semiconductor electrode of Si, Ge, ZnO, or Cds, or electron conductor of titanium. Preferred are glassy carbon electrode and gold electrode. The electrode may be covered with electroconductive polymer film or monomolecular film.

The electrode may be placed on an electro-insulating support material.

The electro-insulating support material can be prepared from glass, ceramics, polymer materials (e.g., polyethylene terephthalate, cellulose acetate, polycarbonate of Bisphenol A, polystyrene, poly(methyl methacrylate), silicon, active carbon, and porous materials (e.g., porous glass, porous ceramics, porous silicon, porous active carbon, cloth, knitted cloth, non-woven cloth, filter paper, and membrane filter). Polymer materials, glass, and silicon are preferably employed.

Generally, the electro-insulating support material is employed in the form of a sheet (or a film). The sheet of the support material preferably has a thickness in the range of 100 to 1,000 μm.

The nucleic acid detective means of the invention is preferably composed of a hydrophobic, electro-insulating support material, a plurality of hydrophobic electrodes placed on the support material, a plurality of peptide nucleic acids fixed on each of the electrodes, and, optionally, a plurality of spacer molecules fixed on each of the electrodes at free areas (i.e., areas where no PNA molecules are present). Each of the electrodes is preferably arranged apart from the adjoining electrodes so that each electrode is insulated from the adjoining electrodes. The electrode may be placed on the support material via an intermediate layer such as a hydrophilic intermediate layer which may have electron charges.

An example of the structure composed of an electro-insulating support material and a plurality of electrodes arranged on the support material is a silicon chip described in Sosnowski, R. G., et al., Proc. Natl. Acad. USA, 94, 1119-1123(1997). The electrode may be produced on a polymer film using a composite sheet of a polymer film and a metal film.

The reactive electrode of the invention preferably comprises an electrode and a plurality of vinylsulfonyl groups or their reactive precursors each of which is fixed onto a surface of the electrode by covalent bonding, optionally, via a linking group.

The vinylsulfonyl group or its reactive precursor is preferably represented by the following formula (1):

-L-SO₂—X (1)

In the formula (1), X is a group of —CR¹═CR²R³ or —CHR¹—C²R³Y. Each of R¹, R² and R³ independently is a hydrogen atom, an alkyl group having 1 to 6 carbon atoms such as methyl, ethyl, n-propyl, isopropyl, n-butyl, or n-hexyl, an aryl group having 6 to 20 carbon atoms such as phenyl or naphthyl, or an aralkyl group having 7 to 26 carbon atoms in which its alkyl group has 1 to 6 carbon atoms such as benzyl or phenethyl.

Y is a group which can be substituted with a nucleophilic reagent such as —OH, —OR⁰, —SH, NH₃, or NH₂R⁰ (R⁰ can be an alkyl group) or which is released by a base in the form of HY. Examples of the groups of Y include a halogen atom, —SO₂R¹³, —OCOR¹², —OSO₃M, or a quaternary pyridinium group. R¹¹ is a hydrogen atom, an alkyl group having 1 to 6 carbon atoms, an aryl group having 6 to 20 carbon atoms, or an aralkyl group having 7 to 26 carbon atoms in which its alkyl group has 1 to 6 carbon atoms. R¹² is an alkyl group having 1 to 6 carbon atoms, or a halogenated alkyl group having 1 to 6 carbon atoms. M is a hydrogen atom, an alkali metal atom, or an ammonium group which may have one or more sustituents.

Representative examples of the groups of X are illustrated below:

[chemical structure]

Preferred are the groups of (X1), (X2), (X3), (X4), (X7), (X8), (X13) and (X14). More preferred are the groups of (X1) and (X2). Most preferred is the group of (X1), that is, a vinylsulfonyl group.

In the formula (1), L stands for a linking group of divalence or multiple valence, which links the group of —SO₂—X to the solid carrier or another linking group attached to the solid carrier. L may be a single bond or a hydrophilic polymer chain. L may be linked to two or more —SO₂—X groups. Examples of linking groups for L include an alkylene group having 1 to 6 carbon atoms, an alicyclic group having 3 to 16 carbon atoms, an arylene group having 6 to 20 carbon atoms, a heterocyclic group having 2 to 20 carbon atoms and 1 to 3 hetero atoms such as N, S, or P, a divalent group such as —O—, —S—, —SO—, —SO₂—, —SO₃—, —NR¹¹— (R¹¹ is a hydrogen atom, an alkyl group having 1-15 carbon atoms, preferably, 1-6 carbon atoms such as methyl or ethyl, an aryl group having 6-20 carbon atoms, or an aralkyl group of 7 to 21 carbon atoms having an alkyl group of 1 to 6 carbon atoms), or —CO—. These linking groups can be present alone. However, the linking groups can be used in combination. Therefore, L can be —NR¹¹—, —SONR¹¹—, —CONR¹¹—, —NR¹¹COO—, and —NR¹¹CONR¹¹—. If two or more of R¹¹ are present in one linking group, these R¹¹ can be combined to form a ring. The alkyl group, aryl group, and aralkyl group for R¹¹ can have one or more substituents such as a hydroxyl group, an alkoxy group having 1 to 6 carbon atoms, an alkenyl group having 1 to 6 carbon atoms, a carbamoyl group having 2 to 7 carbon atoms, an alkyl group having 1 to 6 carbon atoms, an aralkyl group having 7 to 16 carbon atoms, an aryl group having 6 to 20 carbon atoms, a sulfamoyl group (or its salt with Na, K, or other cation), a carboxyl group (or its salt with Na, K, or other cation), a halogen atom, an alkenylene group having 1 to 6 carbon atoms, an arylene group having 6 to 20 carbon atoms, a sulfonyl group, or combinations of these groups and atoms.

Preferred examples of the L of the formula (1) are illustrated below (in which “a” is an integer of 1 to 6, preferably 1 or 2, and “b” is an integer of 0 to 6, preferably 2 or 3):

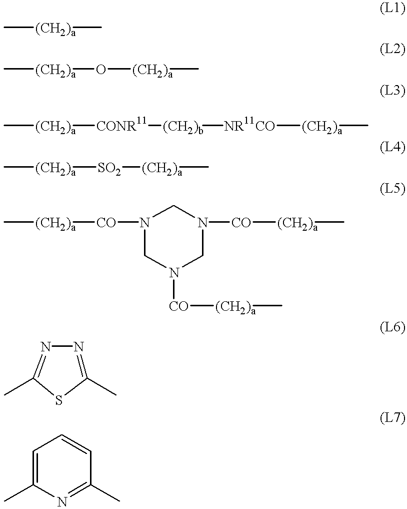

The alkylene group of the above-illustrated divalent or trivalent groups can have a substituent group represented by —SO₂—CH═C₂.

As for the group of “-L-SO₂—X”, the following groups also can be mentioned:

[chemical structure]

The reactive electrode of the invention is preferably produced by bringing an electrode having reactive groups on its surface into contact with disulfone compounds having the following formula (2):

X¹—SO₂-L-SO₂—X (2)

Each of X¹ and X² is one of groups of —CR¹═CR²R³ and —CHR¹—CR²R³Y which are already described for the group of X. L² is a linking group such as one already described for L.

The disulfone compound of the formula (2) can be brought into contact with the solid carrier in the presence of an aqueous medium to prepare a reactive solid carrier of the invention.

Representative examples of the disulfone compounds of the formula (2) are illustrated below.

[chemical structure]

Most preferred is 1,2-bis(vinylsulfonylamide)ethane which corresponds to (S1).

The disulfone compounds can be synthesized in the manners described in Japanese Patent Publications No. 47-2429 and No. 50-35807, and Japanese Patent Provisional Publications No. 49-24435, No. 53-41551 and No. 59-18944.

The reactive electrode of the invention can be also prepared by bringing a solid carrier having reactive groups on its surface into contact with a bifunctional compounds of the formula (3):

X¹—SO₂-L²-X³ (3)

In the formula (3), X¹ is one of groups of —CR¹═CR²R³ and —CHR¹—CR²R³Y which are already described for the group of X. L² is a linking group such as one already described for L.

H³ is a maleimide group, a halogen atom, an isocyanate group, a thioisocyanate group, a succimidoxy group, an aldehyde group, or a carboxyl group.

The bifunctional compound of the above-identified formula can be brought into contact with the solid carrier in the presence of an aqueous medium to prepare a reactive solid carrier of the invention.

[Peptide Nucleic Acid—PNA]

The PNA preferably employable in the invention has the following formula (4):

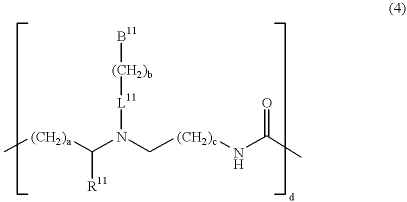

In the formula (4), the symbols of B¹¹, R¹¹, L¹¹, a, b, c, and d have the meanings described below.

B¹¹ is a ligand and represents one of bases of natural nucleic acids (i.e., A, T, C, G, I, or U) or its analogue. B¹¹ is bonded through the 9th position in the case that the base is a purine base such as adenine, guanine or inosine, and through the 1st position in the case that the base is a pyrimidine base such as thymine, uracil or cytosine. The base analogue is an organic base which is similar to the base of natural origin in its chemical structure, for instance, a base group which is prepared by replacing the carbon or nitrogen atom of the purine or H pyrimidine ring with a nitrogen or carbon atom, respectively, or a base group modifying the purine or pyrimidine ring with a substituent such as a sulfhydryl group or a halogen atom. Otherwise, B¹¹ can be an aromatic moiety containing no nucleic acid base, an alkanoyl group having 1 to 4 carbon atoms, a hydroxyl group, or a hydrogen atom. Examples of the base analogues include 7-deazaadenine, 6-azauracil, and 5-azacytosine. B¹¹ also can be a DNA intercalator, a reporter ligand, a protein label such as hapten or biotin, a spin label, or a radioactive label. Particularly preferred are nucleic acid bases (i.e., A, T, C, G, and U).

R¹¹ is a hydrogen atom or a group derived from a side-chain of an α-amino acid of natural origin. Examples of such groups include an alkyl group having 1 to 6 carbon atoms, an aryl group having 6 to 20 carbon atoms, an aralkyl group having an alkyl group of 1 to 6 carbon atoms, a heteroaryl group having 6 to 20 carbon atoms, a hydroxyl group, an alkoxy group having 1 to 6 carbon atoms, an alkylthio group having 1 to 6 carbon atoms, a group of —NR¹³R¹⁴ [each of R¹³ and R¹⁴ independently is a hydrogen atom, an alkyl group having 1 to 3 carbon atoms, an alkoxy group having 1 to 3 carbon atoms, an alkylthio group having 1 to 3 carbon atoms, or a hydroxyl group], and a mercapto group. R¹¹ may form an alicyclic ring or a heterocyclic ring in combination with the carbon atom to which R¹¹ is attached.

L¹¹ is a linking group such as a divalent group represented by the group of —CO— or —CONR¹²— [R¹² is a hydrogen atom, an alkylene group having 1 to 4 carbon atoms, a hydroxyl group, an alkoxy group having 1 to 4 carbon atoms, or an amino group], or an alkylene group having 1 to 4 carbon atoms. The alkoxy group and amino group may have one or more substituents such as alkyl of 1-4 carbon atoms, alkoxy of 1-4 carbon atoms, and hydroxyl.

Each of a, b and c independently is an integer of 0 to 5, preferably 1, and d is an integer of 1 to 60, preferably an integer of 1 to 40.

A particularly preferred PNA fragment has the following formula (5), in which each of B¹¹ and d has the same meaning as described above for the formula (4):

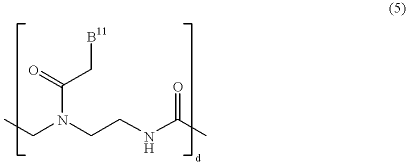

[Mechanism of Fixation of Peptide Nucleic Acid]

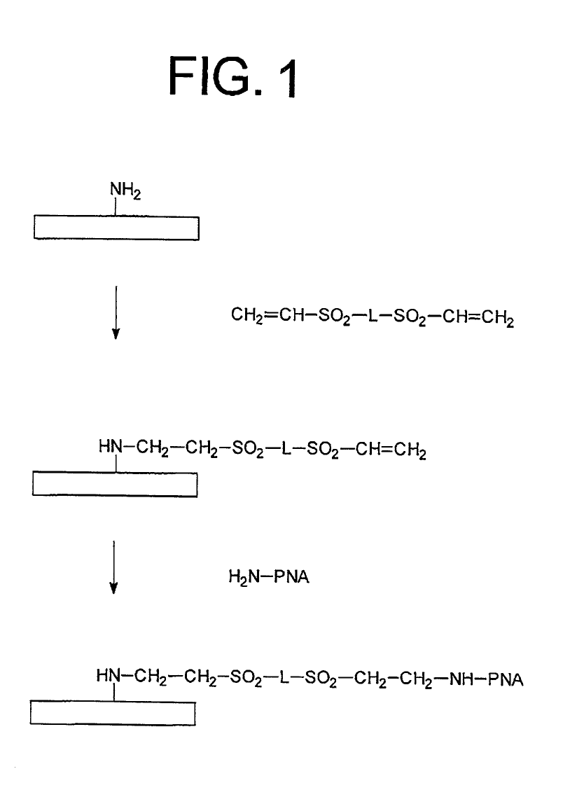

In FIG. 1, a typical process for fixing a peptide nucleic acid (PNA) to an electrode according to the invention is schematically illustrated. In FIG. 1, a reactive compound having at both ends vinylsulfonyl group (i.e., CH₂═CH—SO₂-L-SO₂—CH═CH₂) such as 1,2-bis(vinylsulfonylacetamide)ethane is brought into contact with an amino group attached to an electrode, to give a reactive electrode having a reactive group of -L-SO₂—CH═CH₂ on its surface. The reactive electrode is then brought into contact with a peptide nucleic acid (PNA) having an NH₂ group at its terminal, so that the PNA is fixed to the electrode by covalent bonding.

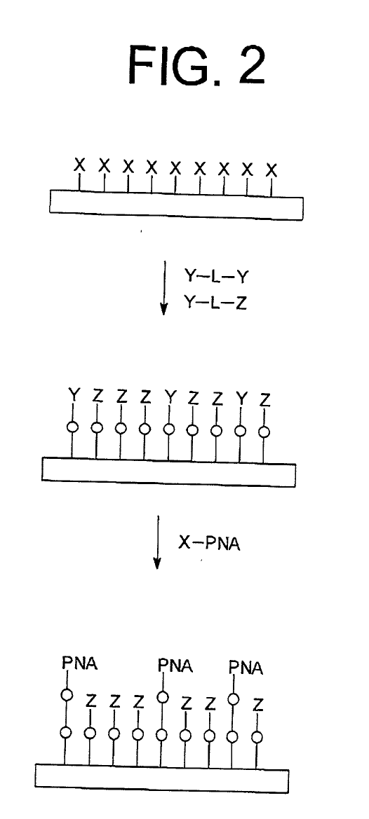

In FIG. 2, a typical process for fixing a peptide nucleic acid (PNA) and a spacer group to an electrode according to the invention is schematically illustrated. In FIG. 2, an electrode having on its surface reactive groups (X) is simultaneously brought into contact with spacer compounds having a group Y which is reactive to the group X at one end and a group Z which is unreactive to the group X, and a reactive compound having at both ends a reactive group Y (typically, vinylsulfonyl group) such as 1,2-bis(vinylsulfonylacetamide)ethane, to give a reactive electrode having on its surface reactive groups Y as well as unreactive groups Z. The reactive electrode is then brought into contact with a peptide nucleic acid (PNA) having a reactive group (such as NH₂ group) at its terminal, so that to the electrode are fixed peptide nucleic acids as well as the spacer groups.

[Procedure of Fixation}

The peptide nucleic acids to be fixed on the electrode are dissolved or dispersed in an aqueous solution. Generally, the aqueous solution is once placed on a plastic plate having 96 or 384 wells, and then spotted onto an electrode using a spotting means.

In order to keep the spotted aqueous solution from evaporating, it is preferred to add a high boiling-point compound to the aqueous solution containing peptide nucleic acids. The high boiling-point compound should be soluble in an aqueous medium, should not disturb hybridization procedure, and preferably has an appropriate viscosity. Examples of the high boiling-point compounds include glycerol, ethylene glycol, dimethylsulfoxide, and a hydrophilic polymer having a low molecular weight (typically, in the range of 10³ to 10⁶) such as polyacrylamide, polyethylene glycol, or poly(sodium acrylate). The high boiling-point compound preferably is glycerol or ethylene glycol. The high boiling-point compound is preferably incorporated into an aqueous nucleotide derivative solution in an amount of 0.1 to 2 vol. %, particularly 0.5 to 1 vol. %. Otherwise, the spotted aqueous solution is preferably kept at under the conditions of a high humidity (such as 90% RH or more) and an ordinary temperature (25 to 50° C.).

The aqueous solution is spotted onto the electrode under the condition that each drop of the solution generally has a volume of 100 pL to 1 μL, preferably 1 to 100 nL. The peptide nucleic acids preferably spotted onto the electrode are in an amount (in terms of number) of 10² to 10⁵/cm². In terms of mol., 1 to 10⁻¹⁵ moles are preferably spotted. In terms of weight, several ng or less of peptide nucleic acids are preferably spotted. The spotting of the aqueous solution is made onto the electrode to form several dots having almost the same shape and size. It is important to prepare these dots to have the same shape and size, if the hybridization is to be quantitatively analyzed. Several dots are formed separately from each other with a distance of 1.5 mm or less, preferably 100 to 300 μm. One dot preferably has a diameter of 50 to 300 μm.

After the aqueous solution is spotted on the electrode, the spotted solution is preferably incubated, namely, kept for a certain period at room temperature or under warming, so as to fix the spotted nucleotide derivatives onto the electrode. In the course of incubation, UV irradiation or surface treatment using sodium borohydride or a Shiff reagent may be applied. The UV irradiation under heating is preferably adopted. It is assumed that these treatments are effective to produce additional linkage or bonding between the electrode and the attached peptide nucleic acids. The free (namely, unfixed) peptide nucleic acids are washed out using an aqueous solution. Thus washed solid carrier is then dried to give a PNA-fixed electrode of the invention.

The PNA-fixed electrode of the invention is favorably employable for monitoring of gene expression, sequencing of base arrangement of DNA, analysis of mutation, analysis of polymorphism, by way of hybridization.

[Spacer Groups]

The PNA-fixed electrode of the invention preferably has a plurality of short chain spacer group having a lipophilic moiety or hydrophilic moiety at a free end which are fixed onto a surface area of the electrode having no DNA fragments thereon.

The short chain of the spacer molecule means that the molecular length of the spacer molecule is sufficiently short, as compared with the length of the PNA molecule fixed onto the electrode in their vicinity.

The spacer molecule preferably has a main skeleton composed of an alkylene group having 1 to 6 carbon atoms and has neither cationic groups nor anionic groups. The spacer molecule may have a cyclic group in the molecular chain or as a substituent. Examples of the cyclic groups include an aryl group having 6 to 12 carbon atoms, a cycloalkyl group having 3 to 6 carbon atoms, and a heterocyclic group containing 1 to 4 hetero atoms (e.g., N, S, O, or P) and 2 to 20 carbon atoms.

The hydrophilic moiety can be attached to the spacer molecule in the terminal position or in the vicinity of the terminal position. One or more hydrophilic moieties such as hydroxyl group may be attached to the spacer molecule.

The other end of the spacer molecule is fixed onto a surface area of the electrode where the peptide nucleic acids are not attached.

Examples of the compounds serving as the spacer molecules include alkylene thiols such as hexane thiol and mercapto alcohols such as mercaptomethanol, 2-mercaptoethanol, 3-mercaptopropanol, 4-mercaptobutanol, 5-mercaptopentanol, 6-mercaptohexanol. Preferred are 6-hexanethiol, 2-mercaptoethanol, 3-mercaptopropanol, 4-mercaptobutanol, 5-mercaptopentanol, and 6-mercaptohexanol.

Onto the electroconductive substrate, two or more different spacer molecules can be provided.

[Hybridization]

The hybridization can be performed essentially in the same manner as that employed in various assay procedures utilizing the conventional DNA chip.

When the electrochemical analysis is performed, an electrochemically active molecule, specifically, an electrochemically active threading intercalator, is preferably employed for insertion into a hybrid formed by the peptide nucleic acids (i.e., probe molecules) and a sample nucleic acid fragment on the electrode. The threading intercalator assists easy flowing of electric current from or to the electrode along the formed hybrid structure. The electrochemical threading intercalator can be present when hybridization takes place. Otherwise, the threading intercalator can be brought into contact with a previously formed hybrid structure. In the latter case, a free nucleic acid sample which is not hybridized with the fixed peptide nucleic acids is preferably removed from the electrode by washing with a mixture of a surfactant (preferably sodium dodecylsulfate) and a buffer (preferably a citrate buffer) in advance of the contact with the intercalator. The intercalator is preferably brought into contact with the hybrid in an aqueous solution at a concentration of 10 nM to 10 mM.

The hybridization is preferably performed at a temperature between room temperature and approximately 70° C., for 0.5 to 20 hours.

[Electrochemically Active Threading Intercalator]

Some electrochemically active threading intercalators are already known. A representative example of the intercalator is a threading intercalator having an electroconductive group at one end or both ends. The threading intercalator having the electroconductive group preferably has an oxidative-reductive activity. The oxidative-reductive activity can be imparted to the threading intercalator by incorporating into the intercalator a ferrocene group, a catechol amine group, a metal bipyridine complex group, a metal phenanthroline complex group, or a viologen group. The intercalator moiety preferably comprises a naphthaleneimide moiety, an anthracene moiety, or an anthraquinone moiety. Preferred electrochemically active threading intercalator is a ferrocene-containing naphthalene diimide compound [NDIFc₂-1, which is prepared from carboxylic acid ester of N-hydroxysuccinimide and a corresponding amine compound, see S. Takenaka et al., J. Chem. Soc. Commun., 1111 (1998)]:

[chemical structure]

A ferrocene-containing naphthalene diimide derivative having the following formula is also preferably employed:

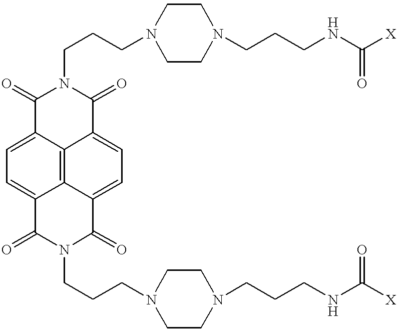

In the above-illustrated formula: X is one of the following ferrocene derivative groups:

[chemical structure]

The threading intercalator having an electroconductive group comprises not only the oxidative-reductive active moiety and the intercalator moiety but also a linker moiety placed between these moieties. The 1,4-dipropylpiperazinyl group of the formula is an example of the linker moiety. The piperazinyl group can be replaced with an quaternary imino group. An intercalator of the below-illustrated formula which has a quaternary imino group always is cationic regardless of pH condition. This means that the intercalator is firmly fixed to the DNA hybrid and PNA hybrid. Accordingly, it is favorably employed in the invention. Particularly, the intercalator having a quaternary imino group is preferred in the use in combination with the PNA chip. The linker can be an N-alkyl group having 1 to 6 carbon atoms (e.g., methyl, ethyl, or n-propyl). The oxidative-reductive potential of the ferrocene moiety of the intercalator varies depending upon the nature of the linker moiety.

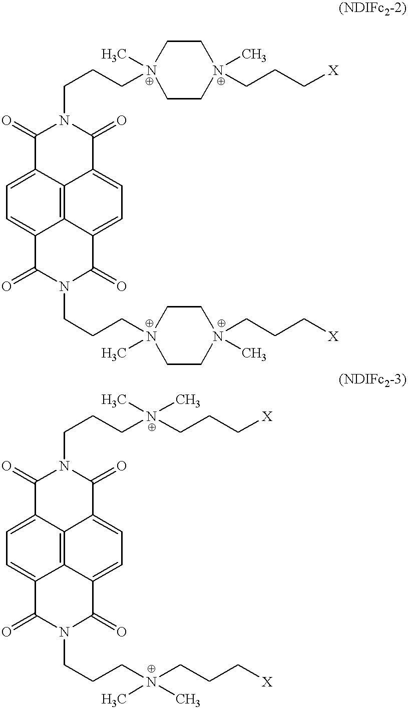

Also preferred are electroconductive threading intercalators which have the formula (6), particularly the formula (7):

Ea-La-X-Lb-Eb (6)

Ea-La-L2a-X-L2b-L1b-Eb (7)

In the formulas (6) and (7), X represents a divalent cyclic group which may have one or more substituents.

The divalent cyclic group preferably is a plane cyclic group. Examples of the divalent cyclic groups include a naphthalene diimide group having two bonding sites at its two nitrogen atoms, an anthracene group having two bonding sites at 2- and 6-positions or 1- and 5- positions (preferably 2- and 6-positions), an anthraquinone group having two bonding sites in the same manner as in the anthracene group, a fluorene group having two bonding sites at 2- and 6-positions, a biphenylene group having two bonding sites at 2- and 6-positions, a phenantholene group having two bonding sites at 2- and 7-positions, and a pyrene group having two bonding sites at 2-and 7-positions. Preferred is a naphthalene diimide group having two bondings at the nitrogen atoms. The substituent can be a halogen atom (e.g., F, Cl, or Br), or an alkyl group having 1 to 6 carbon atoms such as methyl, ethyl, or n-propyl.

In the formula (6), each of La and Lb independently is a group which does not form a conjugated system in combination with the conjugated system of each of Ea and Eb and at least one of which has a site imparting water solubility to the compound or a site that is convertible into a site imparting water solubility to the compound. The site that is convertible into a site imparting water solubility to the compound means such site that it can be converted into a site imparting water solubility to the compound, for instance, by contact with an aqueous acidic solution such as an aqueous sulfuric acid. For instance, an imino group having a methyl substituent can be converted into a site having a sulfate group by contact with sulfuric acid. Thus formed site having a sulfate group imparts to the compound a necessary water solubility. The site can have an electric charge.

The water solubility is required for the compound in the case that the compound functions in an aqueous medium as the threading intercalator.

Each of La and Lb preferably has a hydrocarbyl group (which may have one or more substituents) on the side adjacent to Ea and Eb, respectively. The hydrocarbyl group corresponds to L1a and L1b of the formula (7) and further has a group having atomic elements other than carbon atoms on the side adjacent to X. The latter group corresponds to L2a and L2b of the formula (7) Accordingly, La and Lb are preferably represented by -L1a-L2a- and -L1b-L2b-, respectively.

Each of L1a and L1b preferably is an alkylene group having 1 to 6 carbon atoms or an alkenylene group having 2 to 6 carbon atoms, provided that each group may have one or more substituents. Each of L2a and L2b preferably is a linking group containing N, O or S.

Examples of the substituents for L1a and L1b include hydroxyl, halogen, carboxyl, amino, cyano, nitro, formyl, formylamino, alkyl having 1 to 6 carbon atoms, alkylamino having 1 to 6 carbon atoms, halogenated alkyl having 1 to 6 carbon atoms, cycloalkylamono having 5 to 7 carbon atoms, dialkylamino having 2 to 12 carbon atoms, aryl having 6 to 12 carbon atoms, aralkyl having 7 to 18 carbon atoms which contains alkyl of 1-6 carbon atoms, aralkylamino having 7 to 18 carbon atoms which contains alkyl of 1-6 carbon atoms, alkanoyl having 2 to 7 carbon atoms, alkanoylamino having 2 to 7 carbon atoms, N-alkanoyl-N-alkylamino having 3 to 10 carbon atoms, aminocarbonyl, alkoxycarbonyl having 2 to 7 carbon atoms, heterocyclic ring having 2 to 10 carbo atoms which has 1 to 4 hetero atoms such as S, N, or O, and aryl having 6 to 12 carbon atoms in its ring structure which may have 1 to 5 substituents such as alkyl of 1-6 carbon atoms, alkoxy of 1-6 carbon atoms, or halogen. The number of the substituents preferably is in the range of 1 to 12, more preferably 1 to 3, when the main chain is an alkylene group having 1 to 6 carbon atoms. The number of the substituents preferably is in the range of 1 to 10, preferably 1 to 3, when the main chain is an alkenylene group having 2 to 6 carbon atoms.

Each of L2a and L2b preferably is a linking group containing one or more groups such as an amino bonding, an ester bonding, an ether bonding, a thioether bonding, a diimide bonding, a thiodiimide bonding, a thioamide bonding, an imino bonding, a carbonyl bonding, a thiocarbonyl bonding, or 1,4-piperazinyl bonding, any bonding possibly having one or more substituents. Each of L2a and L2b preferably contains —NHCO— or —CONH—.

Examples of the substituents for L2a and L2b include alkyl having 1 to 3 carbon atoms (e.g., methyl or ethyl), acyl having 2 to 4 carbon atoms (e.g., acetyl), aryl having 6 to 20 carbon atoms (e.g., phenyl or naphthyl), and aralkyl having 7 to 23 carbon atoms which has alkyl of 1-3 carbon atoms (e.g., benzyl).

When L2a or L2b contains an imino bonding, the imino bonding preferably contains a methyl substituent. Accordingly, each of L2a and L2b preferably is N-methyl-di(n-propylenyl)imino or 1,4-di(n-propylenyl)piperazinyl. Most preferred is N-methyl-di(n-propylenyl)imino.

Each of Ea and Eb has oxidation-reduction activity so that each has electroconductivity. It is preferred that each of Ea and Eb independently is a metallocene moiety, a 2,2′-bipyridine complex moiety, a cyclobutadiene moiety, a cyclopentadiene moiety, a 1,10-phenanthroline moiety, a triphenylphosphine moiety, a cathecol amine moiety, and a biologen moiety. Any moieties may have one or more substituents. Preferred are ferrocene moieties which may have one or more substituents. Examples of the substituted ferrocene moieties are illustrated below.

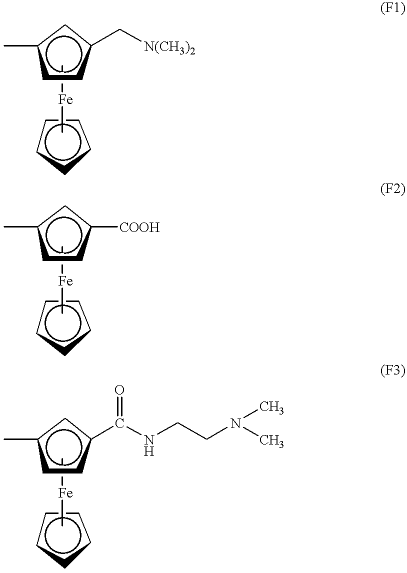

In the above-illustrated substituted ferrocene moieties, the substituent may be present in other positions on the cyclopentadienyl group.

The compound of the formula (6) or (7) which is favorably employed as an electroconductive threading intercalator can be prepared by a process similar to the process described in the aforementioned Japanese Patent Provisional Publication No. H9-288080.

Alternatively, the compound of the invention can be efficiently synthesized from a known diamine compound in accordance with the following synthesis route:

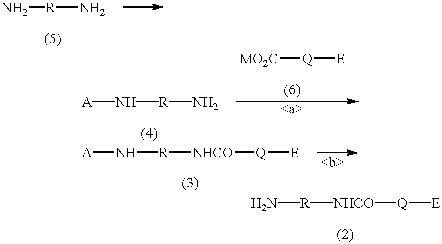

[Electroconductive Cationic Compound]

The complementary nucleic acid detective means of the invention contains non-ionic peptide nucleic acid probe molecules. If complementary nucleic acids having negative charge are bonded to the peptide nucleic acid probe molecules by hybridization, the hybridized probe molecules only become to have negative charge. Therefore, if a cationic compound having an electroconductive label is present in an aqueous medium in which the hybridization reaction proceeds, the cationic compound is predominantly attached to the resulting hybrid complex. This means that the hybrid complex can be located by electrochemically detecting the electroconductive label attached to the electrode.

The electroconductive cationic compound can be a monomer having a cationic group and an electroconductive moiety, or a polymer having a cationic group and an electroconductive moiety. The polymer can be a polymer of a monomer having an cationic group and an electroconductive moiety, a copolymer of a monomer having an cationic group and a monomer having an electroconductive moiety, or a copolymer of a monomer having an cationic group and an electroconductive moiety and a comonomer.

The polymer having a cationic group and an electroconductive moiety can be produced by known methods. For instance, an ethylenic unsaturated monomer having an electroconductive group and/or a cationic group can be polymerized by radical polymerization to give a desired polymer. The electroconductive group and cationic group can be introduced into the polymer after a polymer having neither electroconductive group nor cationic group is produced. In the latter case, the polymer can be a polymer of natural origin such as starch, dextran, or agarose, gelatin.

Examples of the monomers employable for the preparation of the above-identified polymers include acrylic acid, methacrylic acid, their esters.

The cationic groups are preferably incorporated into the polymer as side chains, and are more preferably attached to side chains of the polymers. Examples of the cationic groups include ammonium cationic group, oxonium cationic groups, sulfonium cationic groups, selenonium cationic groups, arsonium cationic groups, antimonium cationic groups, chloronium cationic groups, bromonium cationic groups, and iodonium cationic groups. Most preferred is an ammonium cationic group. The ammonium cationic group may be a heterocyclic nitrogen-containing group such as morphonium group, pyridinium group, pyrrolinium group, imidazolinium group, pyrrazolium group, 2-pyrrolinium group, pyrrolidium group, 2-imidazolidinium group, piperidinium group, or indolinium group.

The electroconductive label can contain an organic metal compound or an electroconductive group having no metal atoms.

Two or more electroconductive labels giving an electric current at different potentials can be attached to the cationic compound.

The organic metal compound preferably is a π-complex in which a surrounding ligand can supply electron to the center metal atom as well as an electron is supplied from the orbit of the metal atom to the vacant orbit of the ligand. A typical example of the π-complex is a metallocene having the following formula:

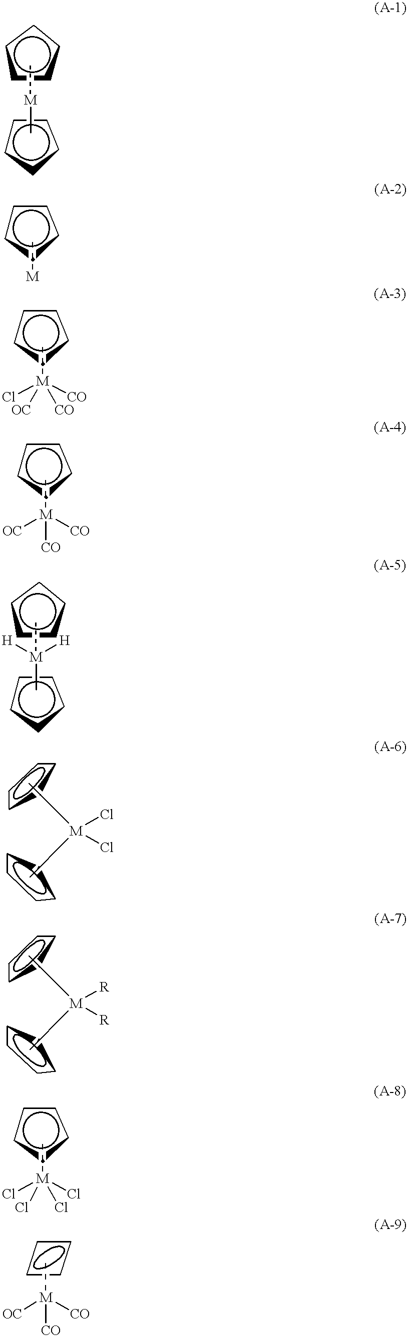

In the above-illustrated formula, the metal atom (M) of the metallocene can be Fe, Ni, Co, Mo, Zn, Cr, Tl, Ta, Ti, Cu, Mn, W, V, Ru or Os Most preferred is Fe.

Other examples of the π-complexs include cyclobutadienyl complex, cyclopentadienyl complex, phenanthroline complex, bipyridyl complex, and triphenylphosphine complex.

Examples of the phenanthroline complexes include tris(phenanthroline) zinc complex, tris(phenanthroline) ruthenium complex, tris(phenanthroline) cobalt complex, di(phenanthroline) zinc complex, di(phenanthroline) ruthenium complex, di(phenanthroline) cobalt complex, and phenanthroline platinum complex.

Examples of the bipyridyl complexes include bipyridyl platinum complex, tris(bipyridyl) zinc complex, tris(bipyridyl) ruthenium complex, tris(bipyridyl) cobalt complex, di(bipyridyl) zinc complex, di(bipyridyl) ruthenium complex, and di(bipyridyl)cobalt complex.

Examples of the triphosphine complexes include COCH₃(PPh₃)₃, CoH(N₂) (PPh₃)₃, RuH₂(PPh₃)₄, and RhH(PPh₃)₄. In the formula, Ph stands for phenyl group.

Other examples of the electroconductive complexes include porphyrins such as chlorophyll, vitamin B₁₂, heme, chlorocruoroheme, and chlorophylide.

Examples of the electroconductive compounds having no metal atom include biologen (described hereinbelow) and 2,2′-bipyridine (described hereinbelow), 1,10-phenanthroline, and catecholamine. In the below-described formula, each of R^{A} and R^{B} independently represents an alkyl group having 1 to 6 carbon atoms, an alkoxy group having 1 to 6 carbon atoms, an aryl group having 6 to 12 carbon atoms, or a heterocyclic group having 2 to 12 carbon atoms and 1 to 4 hetero atoms such as N, O, and S.

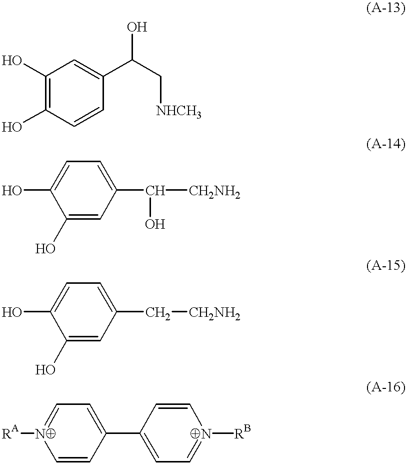

[Sample Nucleic Acid Fragment—Target]

A target DNA fragment (or a sample DNA fragment), which is subjected to the analysis concerning the presence of a complementary DNA fragment can be obtained from various origins. In the analysis of gene, the target DNA fragment is prepared from a cell or tissue of eucaryote. In the analysis of genome, the target DNA fragment is obtained from tissues other than erythrocyte. In the analysis of mRNA, the target sample is obtained from tissues in which mRNA is expressed. If the DNA chip has an oligonucleotide fixed in its solid carrier, the target DNA fragment preferably has a low molecular weight. The target DNA may be multiplied by PCR method.

[Hybridization]

The hybridization is performed by spotting an aqueous sample solution containing a target DNA fragment onto a detective means. The spotting is generally done in an amount of 1 to 100 nL. The hybridization is carried out by keeping the detective means having the spotted sample solution thereon at a temperature between room temperature and 70° C., for 6 to 20 hours. After the hybridization is complete, the detection means is washed with an aqueous buffer solution containing a surface active agent, to remove a free (namely, unfixed) sample DNA fragment. The surface active agent preferably is sodium dodecylsulfonate (SDS). The buffer solution may be a citrate buffer solution, a phosphate buffer solution, a borate buffer solution, Tris buffer solution, or Goods buffer solution. The citrate buffer solution is preferably employed.

[Detection of Hybridization]

The detection method of the invention typically comprises the following steps.

In an aqueous solution, to the detective means having thereon the intercalator-containing or cationic compound-attached hybrid structures is applied a potential, and an electric current flowing from or to the electrode and a separately installed counter electrode along the hybrid structure having the electrochemically label is measured, to detect the hybrid on the electrode.

The measurement of electric current can be performed by any of known methods such as cyclic voltamography (CV), differential pulse voltamography (DPD), and potentiostat. The differential pulse voltamography is most preferred.

[Sensitivity Increased Measurement]

The sensitivity of the electric current measurement for the hybrid structure having the electroconductive label compound can be increased when a combination of a substrate and an oxidase which reacts with the substrate to become a reduction type can be present. The electric current flowing between the electrode and the electroconductive moiety can be multiplied by the electron transfer between the electrode and the oxidase of reduction type.

Examples of the oxidases include glucose oxidase, cholesterol oxidase, uricase, and amine oxidase. The corresponding substrates are glucose for glucose oxidase and cholesterol for cholesterol oxidase.

The present invention is further described by the following examples.

### EXAMPLE 1

### Detection of Complementary DNA Fragment Using Threading Intercalator Having Electroconductive Label—(1)

(1) Synthesis of PNA

PNA-H₂N-Lys-T₁₀-H (hereinafter referred to as PNA-T₁₀ in which T stands for thymine) was synthesized in the manner described in P. E. Nielsen et al., Journal of American Chemical Society, 114, 1895-1897(1992) & 114, 9677-9678 (1992).

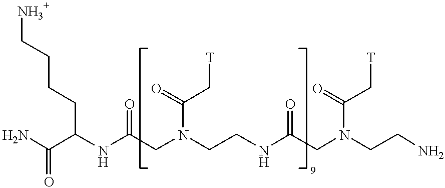

(2) Preparation of PNA-Fixed Gold Electrode

A phosphate buffer solution containing 1,2-bis-(vinylsulfonylacetamide)ethane was spotted on a gold electrode (surface area: 2.25 mm²) having plural mercapto groups on its surface, so as to cause a reaction between 1,2-bis(vinylsulfonylacetamide)ethane and the mercapto group. Thus, 1,2-bis(vinylsulfonylacetamide)ethane was fixed onto the electrode by covalent bonding.

Onto the electrode was then spotted 2 μL of an aqueous solution of PNA-T₁₀ (100 picomol./1 μL), and the spotted solution was kept for one hour at room temperature. Thus, a PNA-fixed gold electrode was prepared.

(3) Preparation of DNA Fragment Sample

DNA-A₁₀ (10 mers of adenine) was prepared in the manner as described in Japanese Patent Provisional Publication No. 9-288080.

(4) Detection of Complementary DNA Fragment

1) Measurement of background value

The PNA-fixed gold electrode prepared in (2) above was immersed at 38° 0C. in an aqueous buffer solution of a ferrocene-labelled threading intercalator having the following formula (50 μm in 0.1 M potassium chloride-0.1 M acetic acid buffer: pH 5.6):

[chemical structure]

To the electrode was applied a voltage from 100 to 700 mV for performing differential pulse voltammetry (DPV). Subsequently, a responsive electric current at an applied voltage of 260 mV (background value) was measured, to give −1.2 μm. The measurement was performed at a pulse frequency of 50 mV, pulse width of 50 mS, and a scanning rate of 100 mV/sec.

2) Detection of complementary DNA fragment

2 μL of 10 mM Tris buffer solution (pH 7.5) containing 70 picomol. of DNA-A₁₀ prepared in (3) above was spotted onto the PNA-fixed electrode prepared in (2) above, and then incubated at 25° C. for 30 minutes. After the incubation was complete, the surface of the electrode was washed with a buffer solution (0.1 M sodium dihydrogen phosphate-disodium hydrogen phosphate, pH 7.0), to remove unfixed DNA-A₁₀.

Subsequently, a responsive electric current at an applied voltage of 260 mV (background value) was measured in the manner described above. The responsive electric current was −2.8 μA. The ratio of variation of the responsive electric against the background value was 133%.

The above-mentioned results indicate that the PNA-fixed electrode in which the PNA probe was fixed by covalent bonding gives a low background value. Further, due to the low background value, the complementary DNA fragment was detected with high sensitivity.

### EXAMPLE 2

### Detection of Complementary DNA Fragment Using Threading Intercalator Having Electroconductive Label—(2)

The procedure of fixing and detecting complementary DNA fragment of Example 1 was repeated except for replacing the intercalator with the following cationic threading intercalator having electroconductive label:

[chemical structure]

The background value, a responsive electric current of the produced hybrid, and a ratio of variation were −1.5 μA, −5.2 μA, and 247%, respectively.

### EXAMPLE 3

### Detection of Complementary DNA Fragment Using Cationic Polymer Having Electroconductive Label

1) Measurement of background value

2 μL of an aqueous solution containing the following ferrocene-containing polyallylamine (approx. 1 nano mol.) was spotted on the PNA-fixed electrode, and the electrode was incubated at 25° C. for 30 minutes.

[chemical structure]

The ferrocene-containing polyallylamine was synthesized by causing a reaction between polyallyamine (available from Nitto Spinning Co., Ltd., molecular weight: approx. 10,000) and ferrocene carboxylic acid in the presence of 1-ethyl-3-(3-dimethylaminopropyl)carbodiimide hydrochloride. The synthesized ferrocene-containing polyallylamine had a molecular weight of approx. 12,000.

After the incubation was complete, the surface of the electrode was washed subsequently with a distilled water and a buffer solution (0.1 M potassium chloride-0.1 M acetic acid, pH 5.6).

To the electrode was applied a voltage from 100 to 700 mV for performing differential pulse voltammetry (DPV). Subsequently, a responsive electric current at an applied voltage of 460 mV (background value) was measured, to give −0.9 μA. The measurement was performed at a pulse frequency of 50 mV, pulse width of 50 mS, and a scanning rate of 100 mV/sec.

2) Detection of complementary DNA fragment

2 μL of 10 mM Tris buffer solution (pH 7.5) containing 70 picomol. of DNA-A₁₀ prepared in (3) above was spotted onto the PNA-fixed electrode prepared in (2) above, and then incubated at 25° C. for 30 minutes. After the incubation was complete, the surface of the electrode was washed with a buffer solution (0.1 M sodium dihydrogen phosphate-disodium hydrogen phosphate, pH 7.0), to remove unfixed DNA-A₁₀.

Subsequently, a responsive electric current at an applied voltage of 260 mV (background value) was measured in the manner described above. The responsive electric current was −2.0 μA. The ratio of variation of the responsive electric against the background value was 122%.

The above-mentioned results indicate that the PNA-fixed electrode in which the PNA probe was fixed by covalent bonding gives a low background value. Further, due to the low background value, the complementary DNA fragment was detected with high sensitivity.

### EXAMPLE 4

### High Sensitive Detection of Complementary DNA Fragment Using Threading Intercalator Having Electro-Conductive Label

In an aqueous buffer solution of a mixture of aqueous 0.1 M acetic acid-potassium acetate solution (pH 5.6) and aqueous 0.1 M potassium chloride solution containing a mixture of the DNA fragment sample (DNA-A₁₀, 268 picomol.), a ferrocene-containing threading intercalator (50 μm) having the below-illustrated formula, glucose (10 mM) and glucose oxidase (originating from Aspergillus niger, available from Wako Co., Ltd.) were placed a trielectrode system composed of the PNA-fixed electrode prepared in Example 1-(2), a platinum electrode, and a silver/silver chloride reference electrode. To the PNA-fixed electrode was applied an electric voltage from 100 to 700 mV at a scanning rate of 10 mV/sec, so as to produce a cyclic voltammogram of the PNA-fixed electrode.

[chemical structure]

The electric current at an applied voltage of 514 mV was −3.2 μA. For comparison, the same procedure was carried out in the absence of glucose and glucose oxidase. In the latter procedure, the electric current at an applied voltage of 514 mV was −0.2 μA. This means that the electric current was multiplied as much as approx. 16 times by the use of glucose and glucose oxidase.

### EXAMPLE 5

### Detection of Complementary DNA Fragment Using PNA-Fixed Electrode Having Spacer Group

(1) Preparation of PNA-Fixed Electrode Having Spacer Group

An aqueous solution (2 μL) containing 6-amino-1-hexanethiol (0.1 mm) and 6-hydro-1-hexanethiol (1 mM) was spotted on a gold electrode (surface area: 2.25 mm²). The spotted solution was left at 45° C. for 2 hours, keeping the spotted solution from rapid evaporation. Thus, reactive groups having an amino group at the free end and spacer groups having a hydroxyl group at the free end were fixed onto the gold electrode. A phosphate buffer solution containing 1,2-bis(vinylsulfonylacetamide)ethane was spotted onto the gold electrode, so as to cause a reaction between 1,2-bis(vinylsulfonylacetamide)ethane and the amino group. Thus, reactive 1,2-bis(vinylsulfonylacetamide)ethane was fixed onto the electrode by covalent bonding under the condition that the spacer groups separated the reactive groups on the electrode.

Onto the electrode was then spotted 2 μL of an aqueous solution of PNA-T₁₀ (300 picomol./1 μL, which was prepared in Example 1), and the spotted solution was kept for one hour at room temperature. Thus, a PNA-fixed gold electrode having spacer groups was prepared.

(2) Measurement of Background Value and Detection of Complementary DNA Fragment

The background value, a responsive electric current of the produced hybrid, and a ratio of variation which were measured in the same manner as in ample 1 were −1.3 μA, −3.9 μA, and 200%, respectively.

(3) Measurement Reproducibility

Ten PNA-fixed gold electrodes having spacer groups were produced in the same manner, and the ten electrodes were subjected to the measurement of background value and the detection of complementary DNA fragment.

The coefficient of variation (CV, N=10) was as low as 6.5%, indicating high measurement reproducibility.

What is claimed is:

1. A nucleic acid detective means comprising an electrode and a plurality of peptide nucleic acids which are fixed onto the electrode via covalent bonding.

2. The detective means of claim 1, wherein the covalent bonding between the electrode and the peptide nucleic acids are produced by the reaction between a reactive hydrogen-containing group attached to the peptide nucleic acid and a vinylsulfonyl group or a reactive precursor thereof attached to the electrode.

3. The detective means of claim 2, wherein the vinylsulfonyl group or reactive precursor thereof is represented by the following formula:
-L-S—X
in which L represents a linking group, and X represents a group of —CR¹═CR²R³ or —CHR¹—CR²R³Y wherein each of R¹, R² and R³ independently is a hydrogen atom, an alkyl group having 1 to 6 carbon atoms, an aryl group having 6 to 20 carbon atoms, or an aralkyl group having 7 to 26 carbon atoms in which its alkyl group has 1 to 6 carbon atoms; Y represents a halogen atom, —SO₂R¹¹, —OCOR¹², —OSO₃M, or a quaternary pyridinium group; R¹¹ is a hydrogen atom, an alkyl group having 1 to 6 carbon atoms, an aryl group having 6 to 20 carbon atoms, or an aralkyl group having 7 to 26 carbon atoms in which its alkyl group has 1 to 6 carbon atoms; R¹² is an alkyl group having 1 to 6 carbon atoms, or a halogenated alkyl group having 1 to 6 carbon atoms; and M is a hydrogen atom, an alkali metal atom, or an ammonium group.

4. The detective means of claim 3, wherein X is a vinyl group having the formula of —CH═CH₂.

5. The detective means of claim 3, wherein L contains a linking atom represented by —NH—, —S—, or —O—.

6. The detective means of claim 3, wherein L is a linking group represented by -(L¹)ₙ—NHCH₂CH₂—, in which L¹ is a linking group and n is 0 or 1.

7. The detective means of claim 1, wherein the electrode comprises gold.

8. The detective means of claim 1, wherein the covalent bonding between the electrode and the peptide nucleic acids are produced by the reaction between a reactive hydrogen-containing group attached to the electrode and a vinylsulfonyl group or a reactive precursor thereof attached to the peptide nucleic acid.

9. A method for detecting a complementary nucleic acid sample which comprises the steps of:
bringing a nucleic acid sample into contact with a detective means which comprises an electrode and a plurality of peptide nucleic acids fixed onto the electrode via covalent bonding in an aqueous medium in the presence of a threading intercalator having an electroconductive moiety to produce a hybrid of the peptide nucleic acid and nucleic acid sample in which the intercalator is intercalated;
applying an electric potential to the electrode of the detective means; and
measuring an electric current flowing between the electrode of the detective means and a separately placed counter electrode through the electroconductive moiety of the intercalator.

10. A method for detecting a complementary nucleic acid sample which comprises the steps of:
bringing a nucleic acid sample into contact with a detective means which comprises an electrode and a plurality of peptide nucleic acids fixed onto the electrode via covalent bonding in an aqueous medium in the presence of a cationic compound having an electroconductive moiety to produce a hybrid of the peptide nucleic acid and nucleic acid sample to which the cationic compound is attached;
applying an electric potential to the electrode of the detective means; and
measuring an electric current flowing between the electrode of the detective means and a separately placed counter electrode through the electroconductive moiety of the cationic compound.

11. A nucleic acid detective means comprising an electrode, a plurality of peptide nucleic acids which are fixed onto the electrode via covalent bonding, and a plurality of spacer groups which are fixed onto the electrode under the condition that the spacers are placed between the peptide nucleic acids.

12. The detective means of claim 11, wherein the covalent bonding between the electrode and the peptide nucleic acids are produced by the reaction between a reactive hydrogen-containing group attached to the peptide nucleic acid and a vinylsulfonyl group or a reactive precursor thereof attached to the electrode.

13. The detective means of claim 12, wherein the vinylsulfonyl group or reactive precursor thereof is represented by the following formula:
-L-SO₂—X
in which L represents a linking group, and X represents a group of —CR¹═CR²R³ or —CHR¹—CR²R³Y wherein each of R¹, R² and R³ independently is a hydrogen atom, an alkyl group having 1 to 6 carbon atoms, an aryl group having 6 to 20 carbon atoms, or an aralkyl group having 7 to 26 carbon atoms in which its alkyl group has 1 to 6 carbon atoms; Y represents a halogen atom, —SO₂R¹¹, —OCOR¹², —OSO₃M, or a quaternary pyridinium group; R¹¹ is a hydrogen atom, an alkyl group having 1 to 6 carbon atoms, an aryl group having 6 to 20 carbon atoms, or an aralkyl group having 7 to 26 carbon atoms in which its alkyl group has 1 to 6 carbon atoms; R¹² is an alkyl group having 1 to 6 carbon atoms, or a halogenated alkyl group having 1 to 6 carbon atoms; and M is a hydrogen atom, an alkali metal atom, or an ammonium group.

14. The detective means of claim 13, wherein X is a vinyl group having the formula of —CH═CH₂.

15. The detective means of claim 11, wherein the spacer group has no electric charge.

16. The detective means of claim 11, wherein the spacer group has a lipophilic moiety at a free terminal thereof.

17. The detective means of claim 11, wherein the peptide nucleic acids and the spacer groups are fixed onto the electrode at a molar ratio in the range of 1:1 to 1:200.

18. A method for detecting a complementary nucleic acid sample which comprises the steps of:
bringing a nucleic acid sample into contact with a detective means which comprises an electrode, a plurality of peptide nucleic acids which are fixed onto the electrode via covalent bonding, and a plurality of spacer groups which are fixed onto the electrode under the condition that the spacers are placed between the peptide nucleic acids in an aqueous medium in the presence of a threading intercalator having an electroconductive moiety to produce a hybrid of the peptide nucleic acid and nucleic acid sample in which the intercalator is intercalated;
applying an electric potential to the electrode of the detective means; and
measuring an electric current flowing between the electrode of the detective means and a separately placed counter electrode through the electroconductive moiety of the intercalator.

19. A method for detecting a complementary nucleic acid sample which comprises the steps of:
bringing a nucleic acid sample into contact with a detective means which comprises an electrode, a plurality of peptide nucleic acids which are fixed onto the electrode via covalent bonding, and a plurality of spacer groups which are fixed onto the electrode under the condition that the spacers are placed between the peptide nucleic acids in an aqueous medium in the presence of a cationic compound having an electroconductive moiety to produce a hybrid of the peptide nucleic acid and nucleic acid sample to which the cationic compound is attached;
applying an electric potential to the electrode of the detective means; and
measuring an electric current flowing between the electrode of the detective means and a separately placed counter electrode through the electroconductive moiety of the cationic compound.

20. A reactive electrode comprising an electrode and a plurality of vinylsulfonyl groups or reactive precursors thereof attached to the electrode.

21. The reactive electrode of claim 20, wherein the vinylsulfonyl group or reactive precursor thereof is represented by the following formula:
-L-SO₂—X
in which L represents a linking group, and X represents a group of —CR¹═CR²R³ or —CHR¹CR²R³Y wherein each of R¹, R² and R³ independently is a hydrogen atom, an alkyl group having 1 to 6 carbon atoms, an aryl group having 6 to 20 carbon atoms, or an aralkyl group having 7 to 26 carbon atoms in which its alkyl group has 1 to 6 carbon atoms; Y represents a halogen atom, —SO₂R¹¹, —OCOR¹², —OSO₃M, or a quaternary pyridinium group; R¹¹ is a hydrogen atom, an alkyl group having 1 to 6 carbon atoms, an aryl group having 6 to 20 carbon atoms, or an aralkyl group hang 7 to 26 carbon atoms in which its alkyl group has 1 to 6 carbon atoms; R¹² is an alkyl group having 1 to 6 carbon atoms, or a halogenated alkyl group having 1 to 6 carbon atoms; and M is a hydrogen atom, an alkali metal atom, or an ammonium group.

22. The reactive electrode of claim 21, wherein X is a vinyl group having the formula of —CH═CH₂.

23. The reactive electrode of claim 21, wherein L contains a linking atom represented by —NH—, —S—, or —O—.

24. The reactive electrode of claim 21, wherein L is a linking group represented by -(L¹)-NHCH₂CH₂—, in which L¹ is a linking group and n is 0 or 1.

25. The reactive electrode of claim 20, wherein the electrode comprises gold.

26. A reactive electrode comprising an electrode, a plurality of vinylsulfonyl groups or reactive precursors thereof attached to the electrode, and a plurality of inert spacer groups which are fixed onto the electrode under the condition that the spacers are placed between the vinylsulfonyl groups or reactive precursors.

27. The reactive electrode of claim 26, wherein the vinylsulfonyl group or reactive precursor thereof is represented by the following formula:
L-SO₂—X
in which L represents a linking group, and X represents a group of —CR¹═CR²R³ or —CHR¹—CR²R³Y wherein each of R¹, R² and R³ independently is a hydrogen atom, an alkyl group having 1 to 6 carbon atoms, an aryl group having 6 to 20 carbon atoms, or an aralkyl group having 7 to 26 carbon atoms in which its alkyl group has 1 to 6 carbon atoms; Y represents a halogen atom, —SO₂R¹¹, —OCOR¹², —OSO₃M, or a quaternary pyridinium group; R¹¹ is a hydrogen atom, an alkyl group having 1 to 6 carbon atoms, an aryl group having 6 to 20 carbon atoms, or an aralkyl group having 7 to 26 carbon atoms in which its alkyl group has 1 to 6 carbon atoms; R¹² is an alkyl group having 1 to 6 carbon atoms, or a halogenated alkyl group having 1 to 6 carbon atoms; and M is a hydrogen atom, an alkali metal atom, or an ammonium group.

28. The reactive electrode of claim 27, wherein X is a vinyl group having the formula of —CH═CH₂.

29. The reactive electrode of claim 26, wherein the spacer group has no electric charge.

30. The reactive electrode of claim 26, wherein the spacer group has a lipophilic moiety at a free terminal thereof.

31. The reactive electrode of claim 26, wherein the peptide nucleic acids and the spacer groups are fixed onto the electrode at a molar ratio in the range of 1:1 to 1:200.

32. The reactive electrode of claim 26, wherein the electrode comprises gold.

33. A method of producing a reactive electrode which comprises bringing an electrode having reactive groups on surface thereof into contact with disulfone compounds having the following formula:
X¹—SO₂-L²-SO₂—X
in which L² represents a linking group, and each of X¹ and X² represents a group of —CR¹═CR²R³ or —CHR¹—CR²R³Y wherein each of R¹, R² and R³ independently is a hydrogen atom, an alkyl group having 1 to 6 carbon atoms, an aryl group having 6 to 20 carbon atoms, or an aralkyl group having 7 to 26 carbon atoms in which its alkyl group has 1 to 6 carbon atoms; Y represents a halogen atom, —SO₂R¹¹, —OCOR¹², —OSO₃M, or a quaternary pyridinium group; R¹¹ is a hydrogen atom, an alkyl group having 1 to 6 carbon atoms, an aryl group having 6 to 20 carbon atoms, or an aralkyl group having 7 to 26 carbon atoms in which its alkyl group has 1 to 6 carbon atoms; R¹² is an alkyl group having 1 to 6 carbon atoms, or a halogenated alkyl group having 1 to 6 carbon atoms; and M is a hydrogen atom, an alkali metal atom, or an ammonium group.
